(19) United States
(12) Patent Application Publication
(10) Pub. No.: US 2026/0020971 A1
(43) Pub. Date: Jan. 22, 2026

(54) Penile Shaping Device

(71) Applicant: Sergio Castaneda, Las Vegas, NV (US)

(72) Inventor: Sergio Castaneda, Las Vegas, NV (US)

(21) Appl. No.: 19/084,744

(22) Filed: Mar. 19, 2025

Related U.S. Application Data
(60) Provisional application No. 63/672,249, filed on Jul. 17, 2024.

(51) Int. Cl. A61F 5/41

(52) U.S. Cl. CPC A61F 5/41; A61F 2005/412; A61F 2005/418

(57) ABSTRACT

An improved penile shaping device includes a vacuum cylinder having a proximal end and a distal end; and one or more penile shaping devices positioned along the length of the vacuum cylinder. The one or more penile shaping devices are configured to create a shaping force on an inserted penis, and can be configured to apply electric current to an inserted penis as well. The invention further includes an attachable angled proximal end and a weighted distal end. The invention is intended to provide a wide variety of therapeutic shaping mechanisms aimed toward correcting penile abnormalities, with numerous treatment methods available.

### CROSS-REFERENCE TO RELATED APPLICATIONS

The present nonprovisional application claims priority benefit of the U.S. provisional application for patent Ser. No. 63/672,249 titled “Penile Shaping Device” filed on Jul. 17, 2024 under 35 U.S.C. § 119(e). The contents of this related nonprovisional application are incorporated herein by reference for all purposes to the extent that such subject matter is not inconsistent herewith or limiting hereof.

### RELATED CO-PENDING U.S. PATENT APPLICATIONS

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### BACKGROUND OF THE INVENTION

### 1. Field of the Invention

One or more embodiments of the invention generally relate to penile shaping devices. More particularly, embodiments of the invention relate to an improved penile straightening, shaping, and enhancement device capable of being implemented for treatment of medical conditions ranging from penile injury to Peyronie's disease.

### 2. Description of the Related Art

Male erectile dysfunction is a common medical problem. It has many roots both physiological and psychological, but its effects may be devastating for both the individual and the individual's partner. Erectile dysfunction and/or penile shortening may be the result of common aging to surgical procedures such as radical prostatectomy and transurethral resection of the prostate.

Peyronie's disease (PD) is a connective tissue disorder involving the tunica albuginea layer of the penis. It is a condition that develops over time, and causes an abnormal curvature of the penis when erect. The fibrotic process that occurs may result in the formation of plaque, one of the classic findings on physical examination of the patient. Men afflicted by this disorder may present with pain, deformity (such as penile curvature or penile shortening) and/or erectile dysfunction (ED).

Several methods of treatment for penile injuries and Peyronie's disease are now in use ranging from drug therapy to the use of medical devices. In addition to erectile dysfunction treatment, many males wish to enhance their penis size and sexual performance, to which analogous treatments are available.

Among the more conventional medical treatment devices commonly used, vacuum enlargement pumps have been known in the art for many years. The manner in which such pumps work is by placing a chamber over a flaccid penis and evacuating the chamber. The evacuation causes a pressure differential between the inside and outside of the chamber. This reduced pressure induces the penis to fill with blood and become erect. Erections may be maintained through the use of constriction bands which prevents blood from leaving the engorged and erect penis.

Most vacuum enlargement pump systems commonly known in the art comprise a chamber having a diaphragm at a lower end thereof and a tube attached at an upper end thereof. The tube is connected to a hand-held vacuum pump device which is usually in the form of an inflatable bulb or a trigger-style having a non-return valve therein. In use, a user places the penis through the diaphragm into the chamber and removes air from the chamber by use of the pump.

Penile traction devices are a class of medical device for treating erectile dysfunction and for increasing penile length. These devices usually consist of a plastic support ring, a silicone band, and two dynamic rods. Such therapy works by holding the penis in a cradle and subjecting it to gentle and progressive straightening forces that can be achieved by the addition of small metal extensions to the dynamic rods and cradle frame every few weeks. As the penile tissues are slowly yet surely stretched, which causes microscopic tears which, in theory, the body heals by producing cells to repair the gap. With each repair phase, the penile tissues expand and form a straighter erect penis.

Penile traction devices have limitations though. Most notably, penile traction devices can be highly uncomfortable and painful to use.

Among the more extreme methods of treatment for penile injury and Peyronie's disease is the use of relatively uncomfortable medical devices which can cause severe discomfort. Surgical procedures and injection of collagenase enzymes are also available. Though proven effective, these methods of treatment come with serious risks which deter numerous men from seeking such treatment; and while surgical therapy has proven itself to be a definitive treatment for penile curvature associated with Peyronie's disease, less invasive options are currently a desired alternative.

Presently, there exists a need for an improved penile shaping device which is capable of providing the results of existing traction devices, but is more comfortable to use and is capable of producing better results. Furthermore, there exists a need for a more versatile penile shaping device which enables a user to synergistically perform multiple different therapies simultaneously.

### SUMMARY

The present invention introduces a new technology, which fulfils the need for a more versatile penile shaping device which provides greater comfort and reduced anxiety for a user. At its essence, the improved penile shaping device comprises a vacuum cylinder having a proximal end and a distal end; and one or more penile shaping devices positioned on the vacuum cylinder, the one or more penile shaping devices configured to create a shaping force on an inserted penis.

Embodiments of the improved penile shaping device can include a kit having a medical-grade plastic vacuum cylinder having a proximal end and a distal end; a plurality of angled medical-grade plastic attachable angled proximal end attachments, the medical-grade plastic attachable angled proximal end attachments. By way of example, and not limitation, a device sold as a kit can include attachments having a zero, forty-five and ninety degree angle, with each angled medical grade plastic proximal end having a fitted opening, the fitted opening capable of creating a sealing flange configured to be sealably held against the pubic area immediately surrounding a penis with the opposite end being attachable to the proximal end of the medical-grade plastic vacuum cylinder.

The improved penile shaping device also includes one or more weighted attachments. The weighted attachments are attachable to the distal end of the vacuum cylinder, which can be used as a weighted distractive force on an inserted penis.

In embodiments of the invention, each of the one or more penile shaping devices positioned on the vacuum cylinder include a shaft and a contact point. Each of the one or more penile shaping devices positioned on the vacuum cylinder extend from the inner diameter of the vacuum cylinder through the vacuum cylinder from the outer diameter of the vacuum cylinder. The contact points can be made from a polymeric material and can be shaped to engage with a penis inserted into the improved penile shaping device.

The improved penile shaping device may also include various electroshock or extracorporeal shock therapeutic implementations. In embodiments of the invention, the contact point is made from an electrically conductive material capable of forming an electrode wherein an electrical current is applied to a penis. The improved penile shaping device and method is versatile and readily implementable to treat a wide variety of penile injury problems and may serve to enhance penis length and girth.

In embodiments of the present invention, the improved penile shaping device can be used with various penile distraction methods. The invention is also capable of being used with a variety of vacuum cylinder devices, thus providing two operational attributes in one device, both a vacuum device and a shaping device.

The improved penile shaping device can be used to perform a wide variety of different therapies. The improved penile shaping device can perform single functions, or it can be used synergistically with other features of the invention to perform multiple therapies.

By way of example, and not limitation, the improved penile shaping device can be simply used as a vacuum pump. A user can simply insert a penis and apply a partial vacuum to the device in order to facilitate and maintain an erection.

Likewise, the penile shaping devices can be used synergistically with a vacuum to provide both a shaping force to an erect penis as well as vacuum therapy. Such use can prove effective in correcting penile shape over time.

The penile shaping devices can be used synergistically with a vacuum and an attachable angled proximal end to provide both a shaping force and an angled force at the base of the penis, as well as vacuum therapy.

Additionally, the penile shaping devices, angled attachable proximal end, and a weighted distal end can be used synergistically with a vacuum to provide increased shaping force to an erect penis in addition to vacuum therapy. Such therapies have been shown to both increase penile size, but to correct abnormalities in penile shape.

In addition, the penile shaping devices can be used synergistically with an insertable sleeve to provide for improved penile shaping. Such a therapy can be used with or without a vacuum and extracorporeal shock therapy.

Furthermore, the penile shaping devices, weighted distal end and electrocorporeal or electroshock devices can be used synergistically with a vacuum to provide increased stimulation to an inserted penis.

In other uses, the electrocorporeal or electroshock devices can be used by itself. Such electroshock stimulation may be sufficient to induce an erection. Other therapies associated with the invention can be utilized synergistically with such devices.

The improved penile shaping device can be implemented as a networkable device. By way of example, embodiments of the invention can be programmed via a smartphone application or as computer software. A user can operate the device via a mobile device or tablet and can store user or patient data. A medical practitioner can access the data to monitor progress.

Use of the improved penile shaping device may involve choosing an attachable proximal end, the proximal end having a varying angle for the penis to easily fit in the device while providing extra clearance for sensitive areas in the pubic region such as the testicles. The user may also choose an attachable and weighted distal end, which attaches to the opposite end of the vacuum cylinder as the proximal end.

Use of the improved penile shaping device involves the user preparing and inserting a penis into a vacuum cylinder, securing the penis in the vacuum cylinder, applying a partial vacuum to engorge a penis, and moving the shaping elements in place so as to straighten a penis. Additional steps include, but are not limited to, applying a mild electrical current for a therapeutic amount of time.

### BRIEF DESCRIPTION OF THE DRAWINGS

The invention directed by way of example, and not by way of limitation, in the figures of the accompanying drawings and in which like reference numerals refer to similar elements and in which:

FIG. 1 is a perspective view of an improved penile shaping device in accordance with an embodiment of the invention;

FIG. 2 is an alternate perspective view of an improved penile shaping device in accordance with an embodiment of the invention;

FIG. 3 is a side elevation view of an improved penile shaping device in accordance with an embodiment of the invention;

FIG. 4 is perspective view of an improved penile shaping device in accordance with an alternative embodiment of the invention;

FIG. 5 is a side elevation view of an improved penile shaping device in accordance with an embodiment of the invention;

FIG. 6 is a top view of six penile shaping devices in accordance with an embodiment of the invention;

FIG. 7 is a top view of an improved penile shaping device in accordance with an embodiment of the invention;

FIG. 8 illustrates a block diagram of a computer system that, when appropriately configured or designed, may serve as a computer system for which the improved penile shaping device, and the components thereof, may be embodied;

FIG. 9 is a perspective view of an improved penile shaping device in accordance with an embodiment of the invention;

FIG. 10 is a side elevation view of an improved penile shaping device in accordance with an embodiment of the invention;

FIG. 11 is a perspective view of a sleeve configured for use in an improved penile shaping device in accordance with an embodiment of the invention;

FIG. 12 is a perspective view of an improved penile shaping device in accordance with an embodiment of the invention; and

FIG. 13 is a side elevation view of an improved penile shaping device in accordance with an embodiment of the invention.

Unless otherwise indicated illustrations in the figures are not necessarily drawn to scale.

### DETAILED DESCRIPTION OF THE PREFERRED EMBODIMENTS

Terminology used herein is used for the purpose of describing particular embodiments only, and is not intended to limit the scope of the present invention. It must be understood that as used herein and in the appended claims, the singular forms “a,” “an,” and “the” include the plural reference unless the context clearly dictates otherwise. For example, a reference to “an element” is a reference to one or more elements and includes all equivalents known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Language that may be construed to express approximation should be so understood unless the context clearly dictates otherwise.

Unless defined otherwise, all technical and scientific terms used herein have the same meanings as commonly understood by a person of ordinary skill in the art to which this invention belongs. Preferred methods, techniques, devices, and materials are described. But any methods, techniques, devices, or materials similar or equivalent to those described herein may be used in the practice or testing of the present invention. Structures described herein should also be understood to refer to functional equivalents of such structures.

References to “one embodiment,” “an embodiment,” “various embodiments,” etc., may indicate that the embodiment(s) of the invention so described may include particular features, structures, or characteristics. However, not every embodiment necessarily includes the particular features, structures, or characteristics. Further, repeated use of the phrase “in one embodiment,” or “in an exemplary embodiment,” do not necessarily refer to the same embodiment although they may. A description of an embodiment with several components in communication with each other does not imply that all such components are required. On the contrary, a variety of optional components are described to illustrate the wide variety of possible embodiments of the present invention.

As is well known to those skilled in the art, many careful considerations and compromises typically must be made when designing for the optimal manufacture of a commercial implementation of such an improved penile shaping device. A commercial implementation in accordance with the spirit and teachings of the invention may be configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art.

The exemplary improved penile shaping device will now be described in detail with reference to embodiments thereof as illustrated in the accompanying drawings.

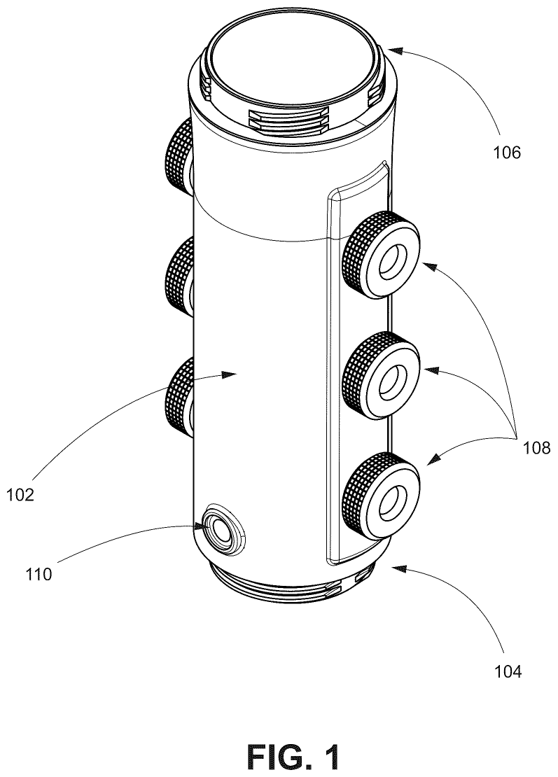

FIG. 1 is a perspective view of the exemplary improved penile shaping device 100 in accordance with an embodiment of the invention. At its core, the improved penile shaping device comprises a vacuum cylinder 102 having a proximal end 104 and a distal end 106. The vacuum cylinder has a vacuum attachment 110 which a vacuum pump can be attached. Such a vacuum cylinder is made from medical grade plastics capable of withstanding partial vacuum forces and penile shaping devices. The vacuum cylinder has a proximal end which is capable of interfacing with the human body, particularly the pubic area which surrounds a human penis.

The system includes a plurality of penile shaping devices having handles 108. The penile shaping devices are placed through the outer diameter of the vacuum cylinder and extend through the inner diameter of the vacuum cylinder 102.

In the preferred embodiment of the invention, the components are made of a clear, medical-grade polycarbonate resin plastic. However, persons skilled in the art will readily appreciate that alternative embodiments may be made from a number of materials such as, but not limited to, clear acrylic plastics, ABS plastic, polycarbonates, polyvinyl chloride (PVC), or various other plastic equivalents.

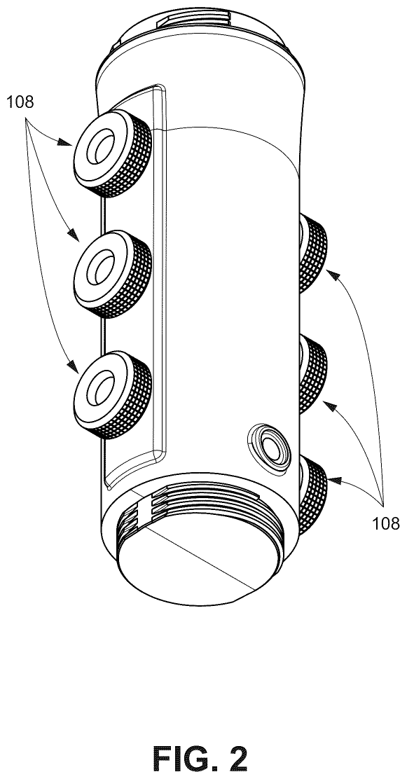

FIG. 2 is an alternate perspective view of an improved penile shaping device in accordance with an embodiment of the invention. The exemplary improved penile shaping device comprises a vacuum cylinder 102 having a proximal end 104 and a distal end 106. The vacuum cylinder has a vacuum attachment 110 which a vacuum pump can be attached. Such a vacuum cylinder is made from medical grade plastics capable of withstanding partial vacuum forces and penile shaping devices. The vacuum cylinder has a proximal end which is capable of interfacing with the human body, particularly the pubic area which surrounds a human penis.

The system includes a plurality of penile shaping devices having handles 108. The penile shaping devices are placed through the outer diameter of the vacuum cylinder and extend through the inner diameter of the vacuum cylinder 102.

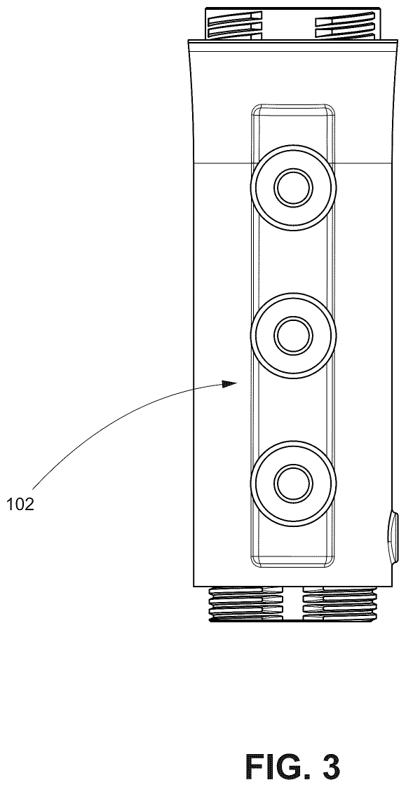

FIG. 3 is a side elevation view of an improved penile shaping device in accordance with an embodiment of the invention. The exemplary improved penile shaping device comprises a vacuum cylinder 102 having a proximal end 104 and a distal end 106. The vacuum cylinder has a vacuum attachment 110 which a vacuum pump can be attached. Such a vacuum cylinder is made from medical grade plastics capable of withstanding partial vacuum forces and penile shaping devices. The vacuum cylinder has a proximal end which is capable of interfacing with the human body, particularly the pubic area which surrounds a human penis.

The system includes a plurality of penile shaping devices having handles 108. The penile shaping devices are placed through the outer diameter of the vacuum cylinder and extend through the inner diameter of the vacuum cylinder 102.

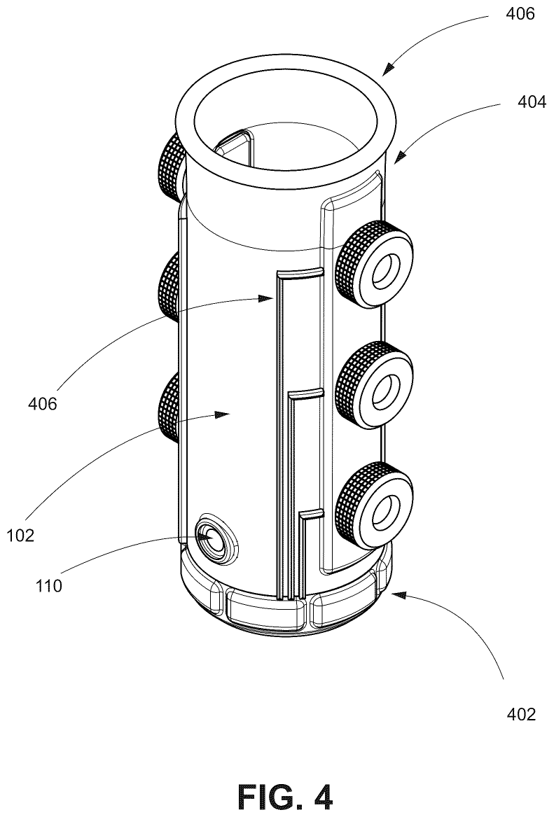

FIG. 4 is perspective view of an improved penile shaping device in accordance with an alternative embodiment of the invention. The exemplary improved penile shaping device comprises a vacuum cylinder 102 having a proximal end 104 and a distal end 106. The vacuum cylinder has a vacuum attachment 110 which a vacuum pump can be attached. Such a vacuum cylinder is made from medical grade plastics capable of withstanding partial vacuum forces and penile shaping devices. Embodiments of the invention further comprise an attachable angled proximal end 404 with a fitted opening 406, and a weighted attachable distal end 402. Both the attachable angled proximal end 404 and the weighted attachable distal end 402 attach to the vacuum cylinder 102 via numerous mechanisms known and appreciated in the art. However, as shown in the drawings, a screw mechanism is a preferred means of attachment.

The attachable angled proximal end is cylindrically shaped, yet angled. In the preferred embodiment of the invention, the attachable angled proximal end 404 is made of a clear, medical-grade polycarbonate resin plastic. However, persons skilled in the art will readily appreciate that alternative embodiments may be made from a number of materials such as, but not limited to, clear acrylic plastics, ABS plastic, polycarbonates, polyvinyl chloride (PVC), or various other plastic equivalents. In one embodiment of the invention, a kit is provided with a forty-five degree angled proximal end attachment, a ninety-degree proximal end attachment, and a one-hundred and eighty degree attachment. A one-hundred and eighty degree attachment is shown in this figure. The purpose of the angled attachments is to allow the user differing angles of shaping therapy coupled with preventing painful stress on the testicles and other sensitive areas.

In the preferred embodiment, the attachable weighted distal end is cylindrically shaped and made of a plastic housing which may made of a clear, medical-grade polycarbonate resin plastic. However, persons skilled in the art will readily appreciate that alternative embodiments may be made from a number of materials such as, but not limited to, clear acrylic plastics, ABS plastic, polycarbonates, polyvinyl chloride (PVC), or various other plastic equivalents. In other embodiments of the invention, the weighted distal ends can be made from a solid metal material such as cast iron or steel. The plastic attachable weighted distal end may be hollow with a fixed amount of a dense material added to create a fixed weight for distraction therapy. Alternative embodiments may assume different shapes, and may be made from other materials such as, but not limited to, wood, metal or stone. In one embodiment of the invention, the weighted distal end attaches to the end of the main cylinder by a basic threaded screw method with the female threaded component located on one end. Persons skilled in the art will readily appreciate that other mechanical means for attaching the components to one another may be used such as, but not limited to, a bayonet mount or other grooved fittings. On the opposite end, numerous accessories may be attached. In this view, a utility hook for hanging extra weight is depicted. However, in other embodiments of the invention, accessories such as, but not limited to, loops, lights, pump mounts, battery mounts may be attached. In an alternative embodiment of the invention, the opposite end of the distal end may also be threaded so as to accommodate additional weighted units to create a greater distractive force when in use.

In some embodiments of the invention, the weighted distal end can house a vacuum pump, a computer system, and a power supply in addition to weighted matter. The power supply provides electrical power for the computer system and vacuum pump. In such embodiments, a user can better manipulate the penile shaping device and perform multiple shaping functions at once.

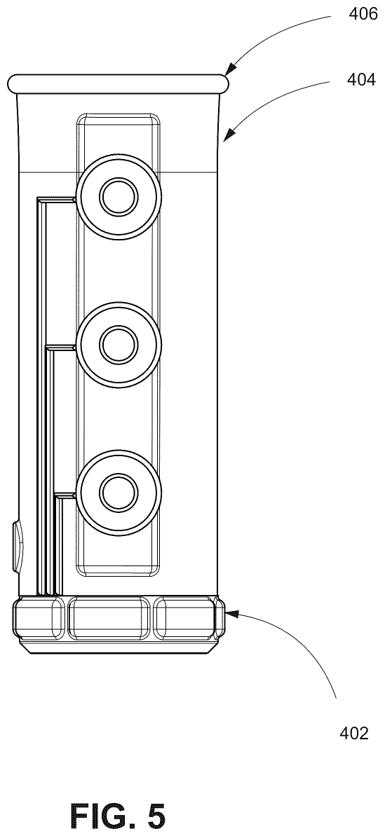

FIG. 5 is a side elevation view of an improved penile shaping device in accordance with an embodiment of the invention. The exemplary improved penile shaping device comprises a vacuum cylinder 102 having a proximal end 104 and a distal end 106. The vacuum cylinder has a vacuum attachment 110 which a vacuum pump can be attached. Such a vacuum cylinder is made from medical grade plastics capable of withstanding partial vacuum forces and penile shaping devices. Embodiments of the invention further comprise an attachable proximal end 404 with a fitted opening 406, and a weighted attachable distal end 402. Both the attachable proximal end 404 and the weighted attachable distal end 402.

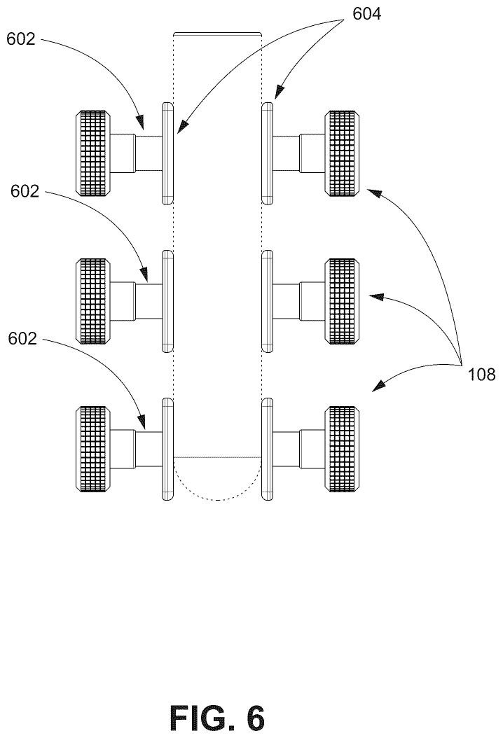

FIG. 6 is a top view of six penile shaping devices in accordance with an embodiment of the invention. Embodiments of the invention include a plurality of penile shaping devices having handles 108 which can be rotated as part of a screw mechanism or depressed as part of a spring mechanism. The penile shaping devices are positioned on the vacuum cylinder extend from the inner diameter of the vacuum cylinder through the vacuum cylinder from the outer diameter of the vacuum cylinder. Each of the one or more penile shaping devices positioned on the vacuum cylinder include a shaft 602 and a contact point 604.

In embodiments of the invention, the number of penile shaping devices can vary. By way of example, and not limitation, a physician or medical practitioner can place penile shaping devices along the vacuum cylinder so as to provide precise shaping locations depending on patient need. In other embodiments, one or more penile shaping devices can be pre-placed along the outer diameter of the vacuum cylinder. As shown in FIG. 6, three penile shaping devices are placed along the height of the vacuum cylinder and are equidistant from one another, with another three penile shaping devices located one hundred and eighty degrees apart from the first three penile shaping devices.

The penile shaping devices are placed through the outer diameter of the vacuum cylinder and extend through the inner diameter of the vacuum cylinder. In some embodiments of the invention, the shaft is threaded and moves laterally and medially from a threaded insertion point via a screw mechanism. In other embodiments of the invention, the shaft moves laterally and medially from a smooth insertion point via a spring mechanism. Persons skilled in the art will appreciate that other mechanisms are readily available to move a shaft laterally and medially in alternate embodiments of the invention. Various types of gasket devices are readily available to provide air-tight seals for such penile shaping devices.

In embodiments of the invention, the contact point is made from a polymeric material and is shaped to engage with a penis inserted into the penile shaping device. Such polymeric materials include plastics that are used in medical devices and are biocompatible, durable, and resistant to corrosion, chemicals, and high temperatures. Such polymeric materials should also be non-absorbent and inert, and able to withstand repeated sterilization cycles.

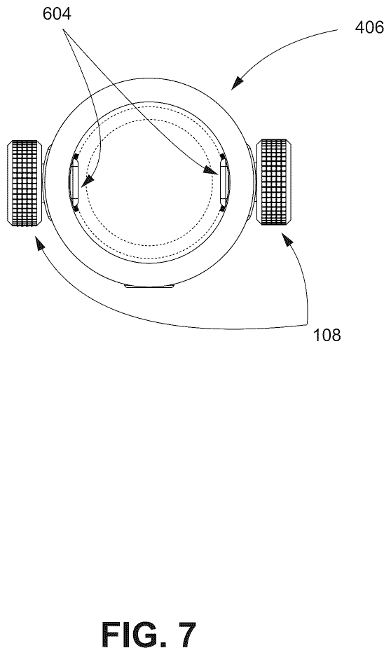

FIG. 7 is a top view of an improved penile shaping device in accordance with an embodiment of the invention. Embodiments of the invention include a plurality of penile shaping devices having handles 108 which can be rotated as part of a screw mechanism or depressed as part of a spring mechanism. The penile shaping devices are positioned on the vacuum cylinder extend from the inner diameter of the vacuum cylinder through the vacuum cylinder from the outer diameter of the vacuum cylinder. In this view, the contact points 604 of the penile shaping devices are shown. Persons skilled in the art will understand that the penile shaping devices move laterally and medially.

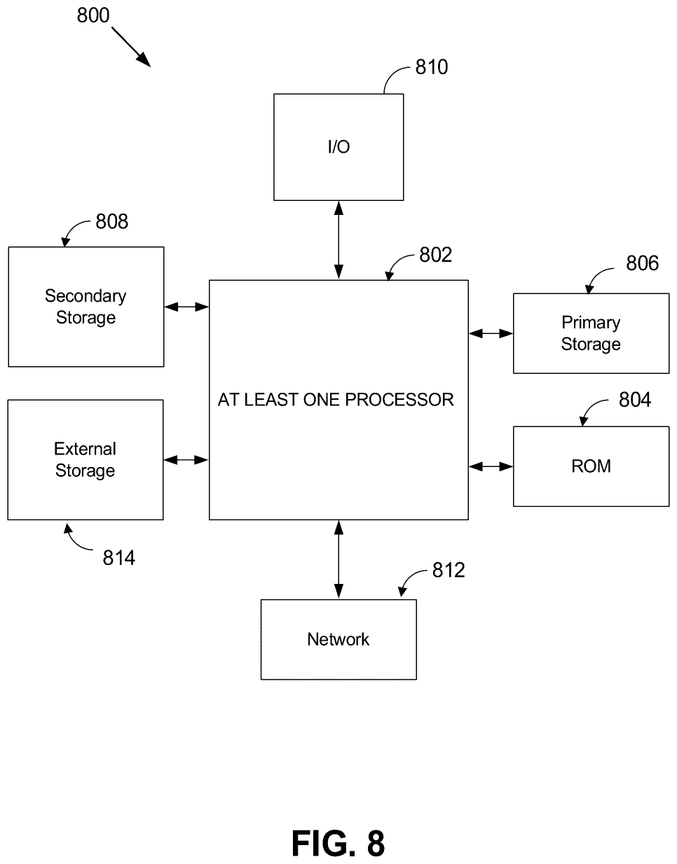

FIG. 8 illustrates a block diagram of a computer system that, when appropriately configured or designed, may serve as a computer system for which the improved penile shaping device, and the components thereof, may be embodied. The computer system 800 includes at least one processor 802 (also referred to as central processing units, or CPUs) that may be coupled to storage devices including a primary storage 806 (typically a random-access memory, or RAM), a primary storage 804 (typically a read-only memory, or ROM). CPU 802 may be of various types including micro-controllers (e.g., with embedded RAM/ROM) and microprocessors such as programmable devices (e.g., RISC or SISC based, or CPLDs and FPGAs) and devices not capable of being programmed such as gate array ASICs (Application Specific Integrated Circuits) or general-purpose microprocessors. As is well known in the art, primary storage 804 acts to transfer data and instructions uni-directionally to the CPU and primary storage 806 typically may be used to transfer data and instructions in a bi-directional manner. The primary storage devices discussed previously may include any suitable computer-readable media such as those described above. A mass storage device 808 may also be coupled bi-directionally to CPU 802 and provides additional data storage capacity and may include any of the computer-readable media described above. Mass storage device 808 may be used to store programs, data and the like and typically may be used as a secondary storage medium such as a hard disk. It will be appreciated that the information retained within mass storage device 808, may, in appropriate cases, be incorporated in standard fashion as part of primary storage 806 as virtual memory. One or more components 810 such as, but not limited to, penile shaping devices, electrodes, sensors, volumeters, etc., can be coupled with the at least one processor so as to provide greater efficiency. A specific mass storage device such as a CD-ROM or flash memory 814 may also pass data uni-directionally to the CPU. In such an embodiment, the computer system 800 can reside in a interactive beverage dispensing system, the instructions for operating each machine being stored as memory in primary storage 804, 806 or mass storage 808. In the preferred embodiment of the invention, a microcontroller system such as, but not limited to, the Raspberry Pi® 3 Model B+. However, other suitable microprocessor and/or microcontroller units can be employed.

The invention may incorporate one or more vibrotactile haptic devices. Such vibrotactile haptic devices can be utilized to provide sexual stimulation or to simply maintain an erection while the other therapies the invention provides.

The CPU 802 may also be coupled to an interface 812 that connects to one or more input/output devices such as track balls, mice, keyboards, microphones, touch-sensitive displays, transducer card readers, magnetic or paper tape readers, tablets, styluses, voice or handwriting recognizers, or other well-known input devices such as, of course, other computers. In the preferred embodiment, the CPU is coupled with a touchscreen monitor. Finally, CPU 802 optionally may be coupled to an external device such as a database or a computer or telecommunications or internet network using an external connection shown generally as a network 814, which may be implemented as a hardwired or wireless communications link using suitable conventional technologies. With such a connection, the CPU might receive information from the network, or might output information to the network in the course of performing the method steps described in the teachings of the present invention.

Persons having skill in the art will appreciate that a CPU unit could be located in the weighted distal end, or could be used as a separate wired unit connected to the vacuum tube. In some embodiments, an application specific integrated circuit (ASIC) could be successfully implemented to perform the functions of the improved penile shaping device. The CPU unit could control an electric vacuum pump located in the weighted distal end as well. Persons skilled in the art will appreciate that numerous vacuum and force sensing devices could also be paired with the CPU unit to provide exact numbers with regards to vacuum applied and weight attached.

The improved penile shaping device can be implemented as a networkable device. By way of example, embodiments of the invention can be programmed via a smartphone application, tablet application, or as computer software. A user can operate the device via a mobile device or tablet and can store user or patient data. In various embodiments, a medical practitioner can access the data to monitor progress and/or change therapies as needed or recommended.

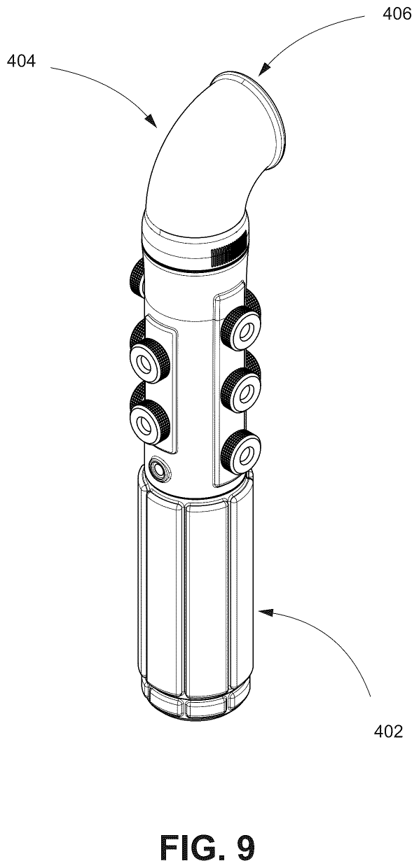

FIG. 9 is a perspective view of an improved penile shaping device in accordance with an embodiment of the invention. Embodiments of the invention comprise an attachable proximal end 404 with a sealing flange 406, and a weighted attachable distal end 402. Both the attachable proximal end 404 and the weighted attachable distal end 402 attach to the vacuum cylinder 102 via numerous mechanisms known and appreciated in the art. However, as shown in the drawings, a screw mechanism is a preferred means of attachment.

The invention is designed to provide the user with a smooth, near-contiguous interior so as to eliminate rough edges which may hurt highly innervated and sensitive tissues. In one embodiment of the invention, the improved penile shaping device may be one single contiguous unit with the angled proximal end and weighted distal end as mere extensions of the vacuum cylinder 102. In another embodiment of the invention, the angled proximal end may be an extension of the vacuum cylinder 102 with the weighted distal end being attachable to the vacuum cylinder 102. In yet another embodiment of the invention, the angled proximal end 404 may attach to the vacuum cylinder 102 with the distal end 402 also attachable to the main vacuum cylinder.

In one embodiment of the invention, a kit is provided with a forty-five degree angled proximal end attachment, a ninety-degree proximal end attachment, and a one-hundred and eighty degree attachment. In one exemplary embodiment, a kit is provided with ten-, twenty-, thirty-, forty-five, sixty-and ninety-degree attachments. The number of angled attachable proximal ends can vary depending on patient need. For example, a doctor can prescribe use of several different types of angled attachments. The purpose of the angled attachments is to allow the user differing angles of distraction therapy coupled with preventing painful stress on the testicles and other sensitive areas. In the preferred embodiment, the attachable angled proximal end 404 is cylindrically shaped, yet angled, and made of a plastic housing which may made of a clear, medical-grade polycarbonate resin plastic. However, persons skilled in the art will readily appreciate that alternative embodiments may be made from a number of materials such as, but not limited to, clear acrylic plastics, ABS plastic, polycarbonates, polyvinyl chloride (PVC), or various other plastic equivalents. A scaling flange 406 may include a sealing constriction ring which is attachably mounted on the angled proximal end in the vicinity of the fitted opening, the sealing flange 406 including a generally ring-shaped body having a circumferentially extending engagement formation for releasable scaling engagement with the angled proximal end in the vicinity of the fitted opening. In the preferred embodiment, the sealing flange 406 will be round shaped and circumferentially shaped the same as the edge of the angled proximal end.

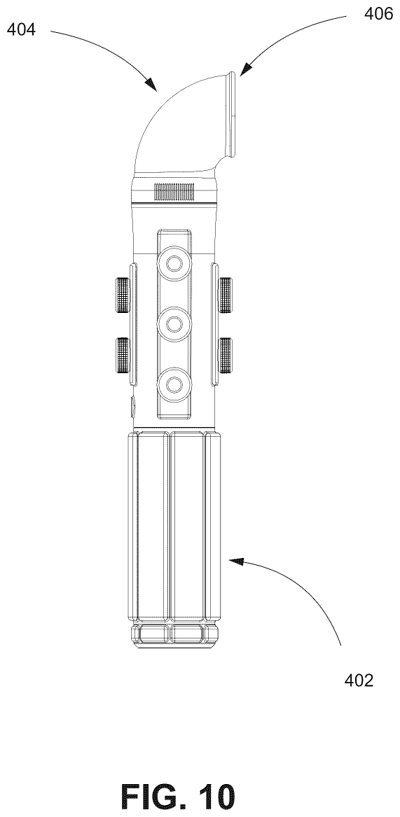

FIG. 10 is a side elevation view of an improved penile shaping device in accordance with an embodiment of the invention. In one embodiment of the invention, the proximal end is angled at ninety degrees. However, persons skilled in the art will appreciate that the proximal end may assume numerous angles depending on patient needs. A scaling flange 406 may include a sealing constriction ring which is attachably mounted on the angled proximal end in the vicinity of the fitted opening, the sealing flange 406 including a generally ring-shaped body having a circumferentially extending engagement formation for releasable sealing engagement with the angled proximal end in the vicinity of the fitted opening. In the preferred embodiment, the sealing flange 406 will be round shaped and circumferentially shaped the same as the edge of the angled proximal end.

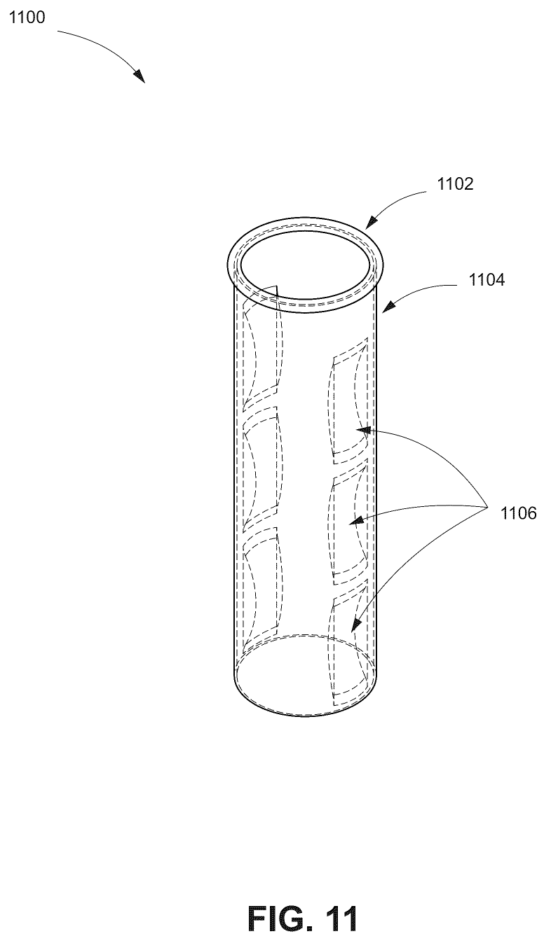

FIG. 11 is a perspective view of a sleeve configured for use in an improved penile shaping device in accordance with an embodiment of the invention. The sleeve 1100 consists of a sealing flange 1102, an elongated tube 1104 and one or more penile shaping points 1106 which are engaged by the one or more penile shaping devices utilized by the penile shaping device. The insertable sleeve 1100 can be shaped and angled in differing configurations so as to create a desired angle for penile shaping. The sleeve 1100 may assume numerous configurations designed for numerous purposes such as, but not limited to, blood flow constriction and sexual stimulation.

In embodiments of the invention, the sleeve is made from a polymeric material and is shaped to engage with a penis inserted into the sleeve penile shaping device. Such polymeric materials include plastics that are used in medical devices and are biocompatible, durable, and resistant to corrosion, chemicals, and high temperatures. Such polymeric materials should also be non-absorbent and inert, and able to withstand repeated sterilization cycles.

In various embodiments of the invention, the sleeve can be used synergistically with various other aspects of the improved penile shaping device. By way of example, and not limitation, the sleeve can be used with or without a vacuum. Moreover, the sleeve can be used to improve shaping forces created by the one or more penile shaping devices.

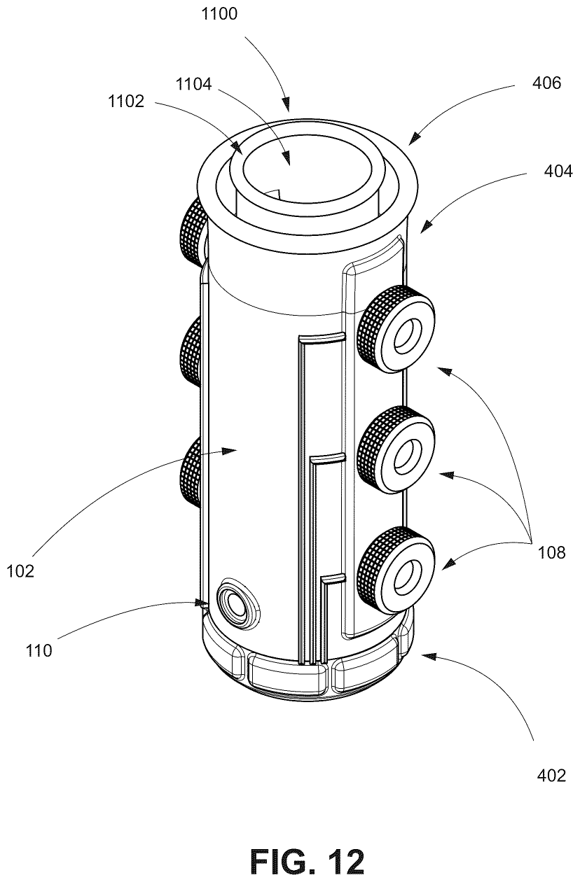

FIG. 12 is a perspective view of an improved penile shaping device in accordance with an embodiment of the invention. In an embodiment of the invention, the improved penile shaping device comprises a vacuum cylinder 102 having a proximal end 104 and a distal end 106. The vacuum cylinder has a vacuum attachment 110 which a vacuum pump can be attached. Such a vacuum cylinder is made from medical grade plastics capable of withstanding partial vacuum forces and penile shaping devices. The vacuum cylinder has a proximal end which is capable of interfacing with the human body, particularly the pubic area which surrounds a human penis. Embodiments of the invention further comprise an attachable proximal end 404 with a fitted opening 406, and a weighted attachable distal end 402. Both the attachable proximal end 404 and the weighted attachable distal end 402. A sleeve 1100 configured for use in an improved penile shaping device in accordance with an embodiment of the invention is inserted in the improved penile shaping device. The sleeve 1100 comprises a scaling flange 1102 and an elongated tube 1104.

The system includes a plurality of penile shaping devices having handles 108. The penile shaping devices are placed through the outer diameter of the vacuum cylinder and extend through the inner diameter of the vacuum cylinder 102.

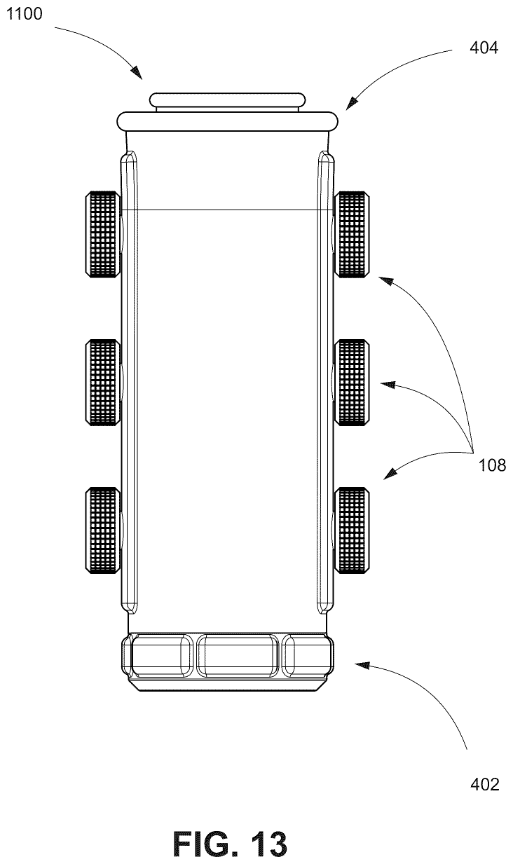

FIG. 13 is a side elevation view of an improved penile shaping device in accordance with an embodiment of the invention.

In some embodiments, the contact points can assume the shape of a sleeve in which a penis can be inserted. In other embodiments, the contact points can be shaped in patient specific fashions in order to provide an optimal amount of shaping force to an engorged penis. A handle 110 can be used to move the shaping means 108 laterally or medially from an inserted penis. One or more gaskets 114 allow for movability of the shaping elements while maintaining a partial vacuum. Persons having skill in the art will appreciate numerous means of implementing a patient-specific shaping element. In embodiments of the invention, the shaping elements can be placed in specific locations on the vacuum cylinder so as to apply shaping pressure to specific areas of an inserted and engorged penis. When such a force is applied over a therapeutic amount of time, a penis can be shaped and the effects of Peyronie's Disease are mitigated.

The improved penile shaping device may also include various electroshock or extracorporeal shock therapeutic implementations 116 to provide increased blood flow and extra muscular tonicity. Such electroshock or extracorporeal shock therapeutic elements can assume numerous shapes and configurations. In some embodiments, the electrotherapeutic elements may be attached to the contact points 112 of the shaping elements 110. The electrotherapeutic elements can be used in conjunction with shaping force applied by the shaping elements. Wiring can be accomplished through sealed apertures in the vacuum cylinder 102. The improved penile shaping device and method is versatile and readily implementable to treat a wide variety of penile injury problems and may serve to enhance penis length and girth.

The improved penile shaping device may also include a patient-specific shaped sleeve 118 which conforms to the shape of a desired penis. Such a sleeve can be made from various materials ranging from, but not limited to, latex or a foam. Persons skilled in the art will readily appreciate the materials available to create such a sleeve are many, and have been proven safe to use. In embodiments of the invention, the sleeve can be shaped by a physician or clinician and prescribed to a patient. Such a sleeve allows a patient to insert a penis and apply both vacuum and shaping therapies. In embodiments of the invention, one or more shaping elements apply shaping forces to specific areas of an engorged penis. Furthermore, such a sleeve can be used as an electrotherapeutic device such that when a small electrical current is applied, both muscular tonicity and blood flow to the penis is enhanced. Other means of electrotherapy are also contemplated as within the scope of this invention.

Use of the improved penile shaping device is designed to be simple and minimally invasive. Use involves the user preparing the device 100, inserting a penis into device and creating a seal with the flange 406 (or 1102) of the invention. Various electrotherapeutic or extracorporeal shock therapeutic elements can be attached to a penis at this time. Once the penis is secured, the user is then able apply a vacuum to create an erect penis. With the penis erect, shaping force is applied to a secured penis with one or more shaping elements. The shaping elements can be moved to shape the erect penis while a vacuum is applied for a therapeutic amount of time. Various electrotherapeutic treatment options can be applied at this time as well. Upon completion of the therapeutic amount of time, or when the user experiences discomfort, the device may be easily detached by loosening the shaping elements and/or releasing vacuum.

The improved penile shaping device can be used to perform a wide variety of different therapies. The improved penile shaping device can perform single functions, or it can be used synergistically with other features of the invention to perform multiple therapies.

By way of example, and not limitation, the improved penile shaping device can be simply used as a vacuum pump. A user can simply insert a penis and apply a partial vacuum to the device in order to facilitate and maintain an erection. For many users, vacuum therapy may be sufficient for one treatment session as part of a greater treatment regimen.

Likewise, the penile shaping devices can be used synergistically with a vacuum to provide both a shaping force to an erect penis as well as vacuum therapy. Such use can prove effective in correcting penile shape over time.

The penile shaping devices can be used synergistically with a vacuum and an attachable angled proximal end to provide both a shaping force and an angled force at the base of the penis, as well as vacuum therapy. Such use can prove effective in correcting penile shape over time.

Additionally, the penile shaping devices, angled attachable proximal end, and a weighted distal end can be used synergistically with a vacuum to provide increased shaping force to an erect penis in addition to vacuum therapy. Such therapies have been shown to both increase penile size, but to correct abnormalities in penile shape.

Furthermore, the penile shaping devices, weighted distal end and electrocorporeal or electroshock devices can be used synergistically with a vacuum to provide increased stimulation to an inserted penis. Such use can prove effective in correcting penile shape over time.

In other uses, the electrocorporeal or electroshock devices can be used by itself. Such electroshock stimulation may be sufficient to induce an erection. Other therapies associated with the invention can be utilized synergistically with such devices. Such use can prove effective in correcting penile shape over time.

Use of the improved penile shaping device may involve choosing an attachable proximal end, the proximal end having a varying angle for the penis to easily fit in the device while providing extra clearance for sensitive areas in the pubic region such as the testicles. The user may also choose an attachable and weighted distal end, which attaches to the opposite end of the vacuum cylinder as the proximal end.

Use of the improved penile shaping device involves the user preparing and inserting a penis into a vacuum cylinder, securing the penis in the vacuum cylinder, applying a partial vacuum to engorge a penis, and moving the penile shaping devices in place so as to straighten a penis. Additional steps include, but are not limited to, applying a mild electrical current for a therapeutic amount of time.

The exemplary improved penile shaping device may be sold as a single unit or as a kit with interchangeable components. In some embodiments, a patient-specific fitted cylinder or custom fitted insert can be made for individual patients. Such a fitted cylinder can be made to work in conjunction with a partial vacuum. For example, differing base units may be made for differing users with differing needs. In other embodiments, differing shaping elements and materials can be utilized. In yet other embodiments, electrotherapeutic devices can be configured to work in conjunction with the improved penile shaping system.

Having fully described at least one embodiment of the exemplary improved penile shaping device, other equivalent or alternative methods of implementing the improved penile shaping device according to the present invention will be apparent to those skilled in the art. Various aspects of the invention have been described above by way of illustration, and the specific embodiments disclosed are not intended to limit the invention to the particular forms disclosed. The particular implementation of the improved penile shaping device may vary depending upon the particular context or application.

By way of example, and not limitation, the improved penile shaping device described in the foregoing patent application is principally directed towards using known shaping means and methods. However, other techniques may instead be applied vacuum pump systems which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Although specific features of the invention are shown in some drawings and not others, persons skilled in the art will understand that this is for convenience. Each feature may be combined with any or all of the other features in accordance with the invention. The words “including,” “comprising,” “having,” and “with” as used herein are to be interpreted broadly and comprehensively, and are not limited to any physical interconnection. Claim elements and steps herein may have been numbered and/or lettered solely as an aid in readability and understanding. Any such numbering and lettering in itself is not intended to and should not be taken to indicate the ordering of elements and/or steps in the claims to be added at a later date.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Any amendment presented during the prosecution of the application for this patent is not a disclaimer of any claim element presented in the description or claims to be filed. Persons skilled in the art cannot reasonably be expected to draft a claim that would literally encompass each and every equivalent.

What is claimed is:

1. An improved penile shaping device comprising:
a vacuum cylinder having a proximal end and a distal end; and
one or more penile shaping devices positioned along the length of the vacuum cylinder, said one or more penile shaping devices configured to create a shaping force on an inserted penis.

2. The improved penile shaping device of claim 1 further comprising an attachable angled proximal end capable of interfacing with the pubic area of a human body, said attachable angled proximal end is attachable to the vacuum cylinder and serves to form a seal between the improved penile shaping device and the pubic area of a human body.

3. The improved penile shaping device of claim 1 further comprising one or more weighted attachments, said weighted attachments attachable to the distal end of the vacuum cylinder.

4. The improved penile shaping device of claim 1 wherein each of the one or more penile shaping devices positioned on the vacuum cylinder include a shaft and a contact point.

5. The improved penile shaping device of claim 1 wherein each of the one or more penile shaping devices positioned on the vacuum cylinder extend from the inner diameter of the vacuum cylinder through the vacuum cylinder from the outer diameter of the vacuum cylinder.

6. The improved penile shaping device of claim 4 wherein the shaft is threaded and moves laterally and medially from a threaded insertion point via a screw mechanism.

7. The improved penile shaping device of claim 4 wherein the shaft moves laterally and medially from a smooth insertion point via a spring mechanism.

8. The improved penile shaping device of claim 4 wherein the contact point is made from a polymeric material and is shaped to engage with a penis inserted into the penile shaping device.

9. The improved penile shaping device of claim 4 wherein the contact point is made from an electrically conductive material capable of forming an electrode wherein an electrical current is applied to a penis.

10. The improved penile shaping device of claim 1 further comprising a computer system having a power supply, at least one electrical output, at least one processor, and memory having computer executable instructions which, when executed by the at least one processor, cause the computer system to send at least one electrical pulse through the one or more penile shaping devices configured to create a shaping force on an inserted penis.

11. An improved penile shaping device comprising:
a vacuum cylinder having a proximal end and a distal end; and
one or more penile shaping devices positioned on the vacuum cylinder, said one or more penile shaping devices positioned on the vacuum cylinder include an electrically conductive shaft and contact point made from an electrically conductive material capable of forming an electrode wherein an electrical current is applied, and configured to create a shaping force on an inserted penis.

12. The improved penile shaping device of claim 11 further comprising an attachable angled proximal end capable of interfacing with the pubic area of a human body, said attachable angled proximal end is attachable to the vacuum cylinder and serves to form a seal between the improved penile shaping device and the pubic area of a human body.

13. The improved penile shaping device of claim 11 further comprising one or more weighted attachments, said weighted attachments attachable to the distal end of the vacuum cylinder.

14. The improved penile shaping device of claim 11 wherein each of the one or more penile shaping devices positioned on the vacuum cylinder extend from the inner diameter of the vacuum cylinder through the vacuum cylinder from the outer diameter of the vacuum cylinder.

15. The improved penile shaping device of claim 11 wherein the shaft is threaded and moves laterally and medially from a threaded insertion point via a screw mechanism.

16. The improved penile shaping device of claim 11 wherein the shaft moves laterally and medially from a smooth insertion point via a spring mechanism.

17. The improved penile shaping device of claim 11 wherein the contact point is made from a polymeric material and is shaped to engage with a penis inserted into the penile shaping device.

18. The improved penile shaping device of claim 11 further comprising a computer system having a power supply, at least one electrical output, at least one processor, and memory having computer executable instructions which, when executed by the at least one processor, cause the computer system to send at least one electrical pulse through the one or more penile shaping devices configured to create a shaping force on an inserted penis.

19. A method of shaping a penis using an improved penile shaping device comprising a vacuum cylinder having a proximal end and a distal end; and one or more penile shaping devices positioned on the vacuum cylinder comprising the steps of:
a. preparing a penis for insertion into the improved penile shaping device;
b. inserting a penis into the vacuum cylinder;
c. securing a penis inserted into the vacuum cylinder;
d. creating a partial vacuum in the vacuum cylinder so as to engorge an inserted penis;
e. moving the one or more penile shaping devices positioned along the length of the vacuum cylinder so as to create one or more shaping forces on an engorged penis; and
f. maintaining the partial vacuum and shaping forces for a therapeutic amount of time.

20. The method of claim 19 further comprising the step of:
a. sending at least one electrical pulse through the one or more penile shaping devices configured to create a shaping force on an inserted penis.
